(19) United States
(12) Patent Application Publication
(10) Pub. No.: US 2009/0305314 A1
(43) Pub. Date: Dec. 10, 2009

(54) Upregulation of Adamts4 Protease Activity for the Treatment of Alzheimer's Disease

(76) Inventors: Dirk Beher, Thousand Oaks, CA (US); Jonathon Wrigley, Cheshire (GB)

Correspondence Address: MERCK AND CO., INC, P O BOX 2000, RAHWAY, NJ, 07065-0907, US

(21) Appl. No.: 12/223,349

(22) PCT Filed: Jan. 30, 2007
(86) PCT No.: PCT/GB2007/050045

§ 371 Date: Nov. 21, 2008

(30) Foreign Application Priority Data
Feb. 1, 2006 (GB) 0601976.4

(51) Int. Cl. G01N 33/53

(52) U.S. Cl. 435/7.92; 435/7.1

(57) ABSTRACT

The invention provides methods of identifying compounds suitable for treating Alzheimer's disease and related conditions by means of modulation of ADAMTS4.

The present invention relates to novel approaches to the treatment of Alzheimer's disease (AD) and related conditions and to the screening of putative drugs against AD.

Alzheimer's disease (AD) is the most prevalent form of dementia. Its diagnosis is described in the Diagnostic and Statistical Manual of Mental Disorders, 4^{th} ed., published by the American Psychiatric Association (DSM-w). It is a neurodegenerative disorder, clinically characterized by progressive loss of memory and general cognitive function, and pathologically characterized by the deposition of extracellular proteinaceous plaques in the cortical and associative brain regions of sufferers. These plaques mainly comprise fibrillar aggregates of β-amyloid peptide (Aβ). Aβ is formed from amyloid precursor protein (APP) via separate intracellular proteolytic events involving the enzymes β-secretase and γ-secretase. Variability in the site of the proteolysis mediated by γ-secretase results in Aβ of varying chain length, e.g. Aβ(1-38), Aβ(1-40) and Aβ(1-42). After secretion into the extracellular medium, Aβ forms initially-soluble aggregates which are widely believed to be the key neurotoxic agents in AD (see Gong et al, PNAS, 100 (2003), 10417-22), and which ultimately result in the insoluble deposits and dense neuritic plaques which are the pathological characteristics of AD. (see Glenner & Long, Biochem Biophys. Res. Commun., 120 (1984) 885-90; Masters et al, PNAS, 83 (1985), 4245-9).

Other dementing conditions associated with deposition of Aβ in the brain include cerebral amyloid angiopathy, hereditary cerebral haemorrhage with amyloidosis, Dutch-type (HCHWA-D), multi-infarct dementia, dementia pugilistica and Down syndrome.

An alternative (non-pathological) processing of APP involves cleavage by an enzyme known as α-secretase in preference to cleavage by β-secretase. Cleavage by α-secretase takes place within the sequence of amino acids that would otherwise form Aβ, and results in fragments having no known pathological effect. Interventions which favour cleavage by α-secretase over cleavage of β-secretase are therefore expected to be of therapeutic effect.

ADAMTS4 and the homologous ADAMTS5 (also known as aggrecanase-1 and -2 respectively) are members of the family of metalloproteinase known as ADAMTS (a dinsintegrrin and metalloproteinase domain with thorombospondin motifs). They are involved in the cleavage of aggrecan, which is a major component of cartilage extracellular matrix, and hence have been extensively studied in connection with possible treatment for arthritis (see, for example, Young et al, Arthritis Res. Ther., 7, (2005), R503-12; Glassson et al, Arthritis Rheum, 50 (2004), 2547-58). Furthermore, selective small-molecule inhibitors of ADAMTS4 have been identified, with a view to treating or preventing arthritis (Yao et al, J. Med. Chem., 44 (2001) 3347-50; Yao et al, Bioorg. Med. Chem. Lett., 12 (2002, 101-4; and WO 99/09000).

In contrast to ADAMTS5, which is expressed almost exclusively in musculoskeletal sites, ADAMTS4 also shows significant levels of expression in the brain and CNS, although its role and effects therein are not well documented. WO 2005/108949 discloses that transfection of APP-expressing cells with ADAMTS4 results in an increased production of Aβ, in particular Aβ (1-42), and therefore advocates the use of compounds or compositions which down-regulate the activity of ADAMTS4 as a treatment for AD.

The present inventors, in contrast, have found evidence which indicates that up-regulation of ADAMTS4 should be of therapeutic benefit.

Thus, in a first aspect, the present invention provides a method for adjusting Aβ production in the brain of a mammalian subject comprising administering to that subject an effective amount of an up-regulator of ADAMTS4 protease activity.

The invention further provides a method for treatment or prevention of a condition associated with deposition of Aβ in the brain comprising administering to a subject in need thereof an effective amount of an up-regulator of ADAMTS4 protease activity.

Also provided is an up-regulator of ADAMTS4 for use in adjusting production of Aβ in the brain of a mammalian subject or for use in treating or preventing a condition associated with deposition of Aβ in the brain.

Diseases associated with the deposition of Aβ in the brain include AD, cerebral amyloid angiopathy, HCHWA-D, multi-infact dementia, dementia pugilistica and Down syndrome, in particular AD. The subject is typically human.

By “up-regulator of ADAMTS4 protease activity” is meant any species which brings about an up-regulation of said protease activity in the brain. Such up-regulation may be the result of a direct interaction of the modulator with ADAMTS4 or its substrate, or alternatively the modulator may interact with one or more of the species controlling the expression of active ADAMTS4 within a cell, for examples by activating one or more genes encoding ADAMTS4 or its precursors.

### DESCRIPTION OF THE DRAWINGS

FIG. 1 illustrates the effect of transfection of ADAMTS4into HEK cells expressing APP695.

FIG. 2 shows the results of SELDI-TOF mass spectrometry analysis of Aβ peptides secreted by HEK APP695 cells transfected with ADAMTS4.

The results disclosed herein indicate that ADAMTS4 influences the processing of amyloid precursor protein (APP) in a complex manner, such that enhancement of the action of ADAMTS4 can be expected to be of therapeutic value.

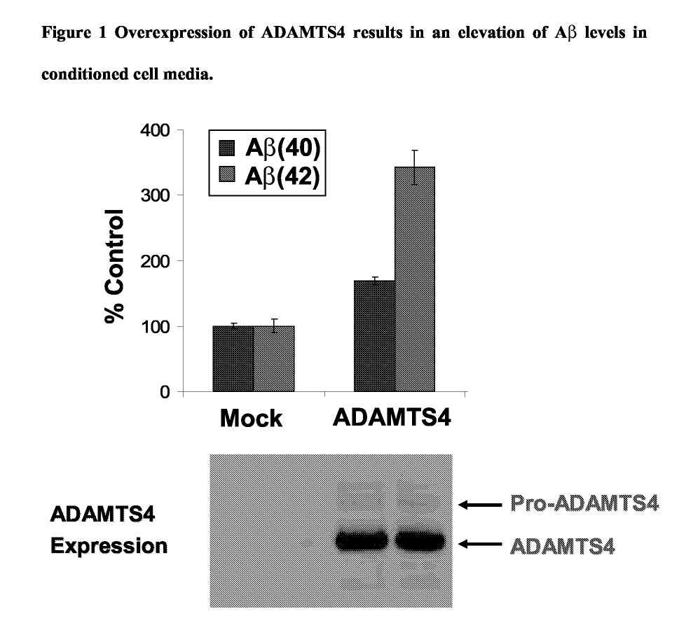

The authors of WO 2005/108949found that transfection of cells expressing APP with ADAMTS4 apparently led to an increase in the levels of Aβ resulting from cleavage of APP by β- and γ-secretase, and that suppression of ADAMTS4 using siRNA techniques led to a decrease n Aβ levels. In those experiments, the Aβ peptides were detected and quantified using an ELISA method (enzyme-linked immunosorbent assay). The present inventors obtained similar results using an essentially similar electrochemiluminescent (ECL) assay (FIG. 1). However, the ELISA and ECL assays both rely on the use of antibodies for capture of the peptides being assayed. Said antibodies typically “recognise” and bind to a particular region of the Aβ peptides, such as the C-terminal region, the N-terminal region or an internal region, and are therefore insensitive to changes elsewhere in the molecule. For example, antibodies which detect the different C-terminal cleavage leading to Aβ (1-40) and Aβ (1-42) will not detect any alteration in the N-terminal cleavage point, and so the exact nature of the species detected is uncertain.

This prompted the present inventors to carry out further experiments using mass spectrometry, specifically the technique of surface enhanced laser desorption/ionisation time-of-flight mass spectrometry (SELDI-TOF), to unambiguously identify and quantify the peptides produced.

This technique involves immunocapture of the peptides using immobilized monoclonal antibodies, followed by laser-induced ionisation and desorption of the captured peptides for mass spectral analysis. (see Davies et al, Biotechniques, 27 (1999), 1258-61, for example).

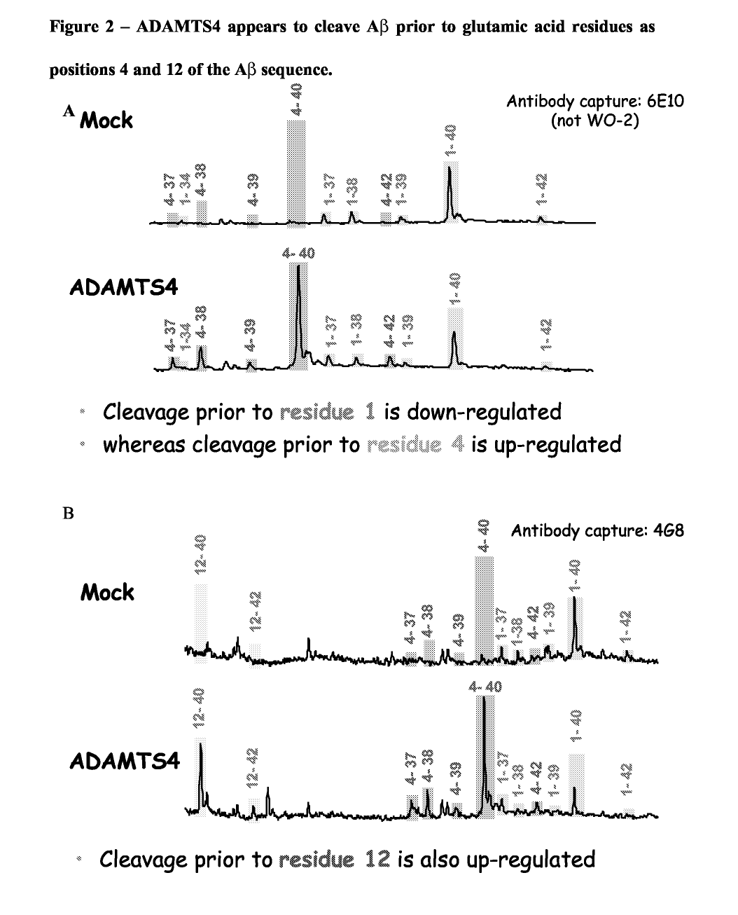

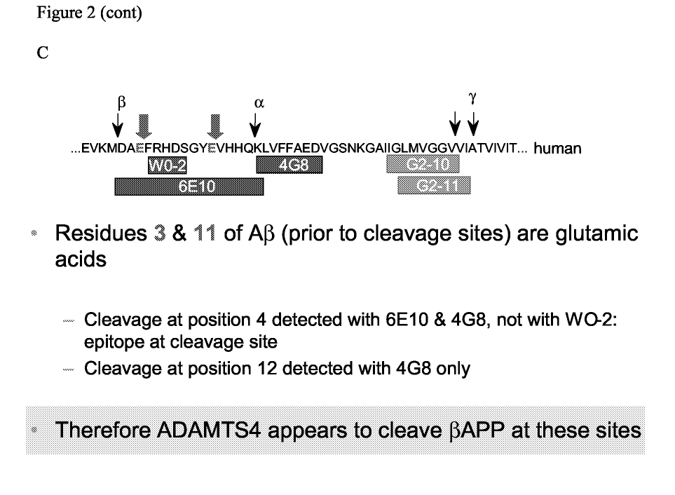

As shown in FIG. 2, the effect of ADAMTS4 transfection is to decrease the production of Aβ (1-40), Aβ (1-42), and other peptides formed by cleavage prior to residue 1 of the Aβ sequences, in favour of production of Aβ (4-40), Aβ (4-42), Aβ (12-40), Aβ (12-42) and other peptides formed by cleavage prior to residue 4 or residue 12 of the Aβ sequence. This is highly significant, as these peptides have no known amyloidogenic properties. For example, Aβ (4-42) is equally prevalent in the brains of AD sufferers and healthy controls. Thus, the effect of ADAMTS4 is apparently to shift production of Aβ from the pathological form to a non-pathological form, and it follows that up-regulation of ADAMTS4 activity will be of therapeutic benefit.

Compounds and compositions capable of up-regulating ADAMTS4 activity may be identified using known techniques. For example, enzyme assays which can identify compounds which modify the activity of ADAMTS4 are published in the literature (eg. Tortorella et al, J Biol, Chem, 275 (2000), 25791-7) and may be used to screen for compounds which enhance said activity. Alternatively, the transfection experiments described herein may be carried out in the presence of test compounds, and their effect on the production of Aβ peptides monitored.

Therefore, according to a further aspect of the invention there is provided a method of screening for compounds suitable for development as a treatment for a condition associated with deposition of Aβ in the brain, said method comprising the steps of:

(a) contacting a test compound with a cell capable of expressing both APP and ADAMTS4;

(b) incubating said cell under conditions consistent with the production of Aβ;

(c) measuring the amounts of Aβ peptides produced; and

(d) identifying whether the presence of the test compound results in one or both of:
(i) a reduction in the levels of one of both of Aβ (1-40) and Aβ (1-42);
(ii) an increase in the levels of any or all of Aβ (4-40), Aβ (4-42), Aβ (12-40) and Aβ (12-42);

relative to levels produced in a control lacking the test compound.

Compounds satisfying step (d) are potentially suitable for treating conditions associated with the deposition of Aβ in the brain, in particular AD.

The measurement in step (c) may be by any suitable means including ELISA, ECL and SELDI-TOF.

In a further aspect, the present invention provides a method for determining whether compounds modulate the expression of Aβ (x-40) and Aβ (x-42) peptides which comprises transfecting cells which express Aβ peptides with ADAMTS4 and measuring the expression of Aβ (x-40) and Aβ (x-42) peptides, where x=1,4 or 12. The measurement may be by any of the detection techniques well known to those skilled in the art (see above), for example SELDI-TOF mass spectrometry. HEK-APP-Notch cells are a suitable source of cells for expressing Aβ peptides.

### EXPERIMENTAL PROCEDURES

Materials

βAPP and its C-terminal fragments were detected using affinity purified polyclonal rabbit antibody R7334 (Beher et al., J Biol. Chem., 276 (2001), 45394-402) (1:2,500 dilution). The secondary antibody used was HRP-conjugated polyclonal goat anti-rabbit F(ab′)₂ fragments (Amersham, 1:5,000 dilution). ADAMTS4 was detected using the monoclonal antibody MS2786 (1:1000)

Cell culture, transfections and cell lines

HEKAPP₆₉₅ cells were maintained under standard conditions (37° C., 5% CO₂) in Dulbecco's Modified Eagles Media (DMEM) supplemented with 10% Foetal Bovine serum (FBS) and the appropriate antibiotic selection (1 μg/ml puromycin). Cells for transfection were plated at a density of 2×10⁶ cells per 10cm³ dish, 24 hours in advance. At the time of transfection, the media on the cells was replaced and transfections were performed using GeneJuice® (Novagen) according to the manufacturer's instructions, optimised as follows. For each dish, 80 μl GeneJuice was added to 2.6 ml serum-free media and either 5 μg of ADAMTS4 DNA was added in a 40 μl volume or 40 μ1 of water was used on identical cells as a control. Following an overnight incubation, the media was again changed and after a further 24 hours, media was collected and cells harvested as required.

ADAMTS4 capture

For capture of ADAMTS4, 100 μl of Heparin Sepharose-6 fast flow resin (Amersham) was incubated with 9 ml conditioned media per reaction (2 hours, rolling table) following equilibration, washing 3 times with TBS (Tris, pH 7.5, 150 mM NaCl). The resin was then centrifuged (4000 rpm, 5 min, 4° C.) and washed again with TBS, which was subsequently removed with a 27.5 gauge needle. Captured proteins were eluted by incubation with SDS-PAGE sample buffer and retention of the supernatant following centrifugation (16,000 rpm, 2 min).

Quantification of Aβ peptides in conditioned cell media

Aβ peptides secreted into the media were quantified using an ECL assay based on a previously described method (Li et al., Nature, 405 (2000) 689-94). Samples of 25 and 50 μl of media were used for detection of Aβ (40) and Aβ (42) respectively, the Aβ (40) samples being supplemented with 25 μl of assay buffer (PBS, 2% BSA, 0.2% Tween-20).The Aβ (40) and Aβ (42) samples were transferred to an Avidin-coated Meso-Scale Discovery plate and supplemented with 25 μl of assay buffer containing 0.16 μg/μl 4G8-Biotin and either 0.04 μg/μl Ruthenylated G2-10 or G2-11 respectively. Following overnight incubation at 4° C., 200 rpm, the media was discarded and the plate washed three times with PBS, prior to addition of 150 μl Meso-S Read Buffer and analysis using the Meso-Scale Discovery analyzer (Sector Imager 6000).

Transient transfection of ADAMTS4 into HEKAPP695 cells results in a significant overexpression of the protein as detected by Western blotting (B.), and a concomitant increase in the levels of both Aβ (40) and Aβ (42) secreted into the cell media (A.) (FIG. 1).

Immunocytochemical analysis of βAPP and ADAMTS4

One day prior to transfection, 13 mm glass coverslips were sterilised, treated with poly-D-lysine, washed with water and left to dry overnight at room temperature. HEKAPP₆₉₅ cells were used to seed four 12 well plates, each at a different cell density (20, 100, 200 and 300×10³ cells per well). Following a five hour incubation, 4 wells on each plate were transfected with 0.1 μg ADAMTS4 DNA, 4 wells with 0.25 μg ADAMTS4 DNA and the remaining 4 wells were subjected to a mock transfection using 2.5 μl water. Transfections were performed as described above, reagents for each of the transfection types being pooled (100 μl serum free media and 1.6 μ1 genejuice into 0.5 μl DMEM per well). Following a 24 hour incubation, the cells were viewed; 200×10³ cells per well were deemed to be optimal and were subjected to the subsequent steps, although 100×10³ cells per well were incubated for a further 24 hours. The cells were then washed twice with PBS, fixed with 4% paraformaldehyde (PFA) for 15 minutes at room temperature and washed twice more. Permeabilization solution (0.5% Triton X-1000) was added to the cells for 15 minutes, followed by 4 washes with PBS and blocking in 10% normal goat serum (NGS, Sigma) for 1 hour at room temperature. Subsequently the cells were incubated overnight at 4° C. with the appropriate antibodies (MS2786, 1:1000 for ADAMTS4; 22C11, 1:1000 for βAPP). Following this incubation, the cells were washed with PBS before the fluorophore-conjugated secondary antibodies (1 in 100 in 10% NGS) were incubated for 1 hour in the dark (anti-rabbit for MS2786 and anti-mouse for 22C11). Subsequent washes with PBS were undertaken before nuclear staining with TOTO-3 iodine (Molecular Probes, 1 in 2000 in PBS for 30 seconds), and two more PBS washes. The coverslips were mounted onto glass slides using Vectashield mounting medium (Vector Laboratories), and images were collected on a Leica confocal microscope using a Leica TCS NT “Image” program version 1.6.587.

Immunocytochemical analysis of ADAMTS4 and PAPP expression in HEKAPP695 cells transfected with ADAMTS4 demonstrate no detectable alteration in PAPP localization despite a significant overexpression of ADAMTS4.

Surface-enhanced Laser Desorption/Ionization Time-of-Flight (SELDI-TOF) Mass Spectrometry analysis of secreted Aβ species

Immunocapture of Aβ peptides from conditioned cell media was performed analogous to previously described methods using the monoclonal antibody 6E10 and purified non-immune mouse IgG as a negative control (Beher et al, J. Neurochem., 82 (2002), 563-75). Briefly, following collection the conditioned media was diluted to 0.5% (v/v) Triton X-100, 25 mM HEPES pH 7.3 (10 ml final volume). Following a centrifugation for 10 min at 4,000 g the supernatant was incubated overnight at 4° C. with the antibody-coupled SELDI protein chip (PS1 preactivated surface ProteinChip array; Ciphergen Biosystems, Fremont) which was processed for SELDI-TOF mass spectrometry as described (Beher et al., supra, 2002).

FIG. 2 shows the results obtained in which:
A—is a SELDI-TOF analysis of Aβ species secreted in conditioned cell media upon transient transfection of HEKAPP695 cells with ADAMTS4. This demonstrates a reduction in Aβ species beginning at position 1 and an elevation in species beginning at position 4.
B—is an analysis with monoclonal antibody 4G8 demonstrates that cleavage at position 12 is also up-regulated.
C—is an analysis of the Aβ sequence and demonstrates that both cleavage at position 4 and 12 correspond to cleavage after a glutamic acid in the Ab sequence, in agreement with previously reported ADAMTS4 cleavage sites. This suggests that ADAMTS4 is responsible for cleaving Aβ at these positions.

1. A method for adjusting Aβ production in the brain of a mammalian subject comprising administering to that subject an effective amount of an up-regulator of ADAMTS4 protease activity.

2. A method for treatment or prevention of a condition associated with deposition of Aβ in the brain comprising administering to a subject in need thereof an effective amount of an up-regulator of ADAMTS4 protease activity.

3. (canceled)

4. A method according to claim 1 wherein the condition associated with the deposition of Aβ in the brain is AD, cerebral amyloid angiopathy, HCHWA-D, multi-infact dementia, dementia pugilistica or Down syndrome.

5. A method of screening for compounds suitable for development as a treatment for a condition associated with deposition of Aβ in the brain, said method comprising the steps of:
(a) contacting a test compound with a cell capable of expressing both APP and ADAMTS4;
(b) incubating said cell under conditions consistent with the production of Aβ;
(c) measuring the amounts of Aβ peptides produced; and
(d) identifying whether the presence of the test compound results in one or both of:
(i) a reduction in the levels of one of both of Aβ (1-40) and Aβ (1-42);
(ii) an increase in the levels of any or all of Aβ (4-40), Aβ (4-42), Aβ (12-40) and Aβ (12-42);
relative to levels produced in a control lacking the test compound.

6. A method according to claim 5 wherein the measurement in step (c) is carried out using ELISA, ECL or SELDI-TOF.

7. (canceled)
